(19) United States
(12) Patent Application Publication
(10) Pub. No.: US 2026/0242314 A1
(43) Pub. Date: Aug. 20, 2026

(54) PROCESS FOR THE PREPARATION OF METHANOL

(71) Applicant: Topsoe A/S, Kgs. Lyngby (DK)

(72) Inventor: Troels Juel Friis-Christensen, Kgs. Lyngby (DK)

(73) Assignee: Topsoe A/S, Kgs. Lyngby (DK)

(21) Appl. No.: 19/489,558

(22) PCT Filed: Jun. 3, 2024
(86) PCT No.: PCT/EP2024/065153

§ 371 Date: Dec. 2, 2025

(30) Foreign Application Priority Data
Jun. 8, 2023 (DK) PA202330073
Jun. 21, 2023 (DK) PA202330091

(51) Int. Cl. C07C 29/151; C25B 1/02; C25B 1/23

(52) U.S. Cl. CPC C07C 29/1518; C25B 1/02; C25B 1/23

(57) ABSTRACT

Process for the preparation of methanol comprising the steps of (a) preparing a hydrogen feedstock by electrolysis (b) providing a carbon oxide feedstock in periods of operating the electrolysis in step (a) (c) mixing at least part of the hydrogen feed and carbon oxide source consisting of carbon monoxide and/or carbon dioxide feed to obtain a methanol synthesis gas; (d) adjusting the molar content of hydrogen, carbon monoxide and/or carbon dioxide from step (c) to a module M of (H2−CO2)/(CO2+CO) to between 1.9 and 2.2 (e) converting the methanol synthesis gas in one or more boiling water reactors to methanol; in periods without operating the electrolysis in step (a) (f) interrupting the converting of the methanol synthesis gas in the one or more boiling water reactors by heat exchange with boiling water, wherein in step (f) the one or more boiling water reactors are heated by one or more auxiliary heaters to maintain boiling of the water in the one or more boiling water reactors.

Methanol is an important chemical feedstock used in a variety of industries including plastics, adhesives, and solvents. Typically, methanol reaction involves the catalytic conversion of carbon oxides and hydrogen gases. In this invention, the methanol reaction is performed in a so-called boiling water reactor.

The boiling water reactor design for methanol synthesis comprises a reactor vessel containing a plurality of tubes loaded with catalyst active for the exotherm methanol synthesis reaction. In the reactor vessel catalyst tubes are installed. The catalyst is typically composed of copper and zinc oxide, which are known to be effective catalysts for the methanol synthesis reaction.

The reactor vessel is equipped with a cooling system that provides cooling to the catalyst bed. The cooling system is designed to maintain the temperature of the catalyst bed within a specified range for the methanol synthesis reaction. The cooling system is preferably a water-cooled system that uses boiling water. The cooling system is a water-cooled system that circulates water through the reactor vessel to remove excess heat.

In operation, the reactants, carbon oxides, and hydrogen gas are introduced into the reactor vessel at a controlled rate. The reactants flow through the catalyst bed, where they are partially converted into methanol. The remaining part is recycled back to the reactor forming a methanol synthesis loop.

The boiling water reactor design for methanol synthesis provides several advantages over traditional methanol production methods.

The main advantage of using boiling water reactor is the efficient removal of reaction energy maintaining the temperature close to equilibrium being more effective.

The production of green methanol, which is methanol that is produced using renewable carbon source and hydrogen generated through electrolysis with a renewable energy source such as wind, solar, or hydro power, faces several challenges that need to be addressed to enable large-scale production and adoption.

During normal operation, the boiling water reactor provides the necessary cooling for the exotherm methanol synthesis reaction. However, due the nature of renewable electricity disruptions in the process, by insufficient supply of hydrogen feedstock, can cause a decrease in the methanol product yields. In some cases, this may even lead to a complete shutdown of the process until the renewable electricity is available again. If the development of reaction heat in the boiling water reactor stops the reactor temperature will gradually reduce because of heat loss eventually requiring a complete shutdown of the methanol synthesis.

The methanol reactor could be maintained hot by its start-up steam ejector using an auxiliary steam supply during hot standby. The steam ejectors are used for heating up the methanol reactors during start-up from cold conditions and same system could also be applied for maintaining it hot. However, it would require the start-up steam system to be online all the time or in case such system does not exist the electrical heater could also take the function as start-up heater.

To prevent such disruptions from impacting the methanol synthesis process, it is necessary to maintain the temperature of the reactor coolant at a constant level. This can be achieved by providing auxiliary heating to the reactor coolant, in addition to the heat provided by the boiling water reactor. Auxiliary heating can help maintain the temperature of the reactor coolant at a safe level, ensuring the efficient and continuous operation of the methanol synthesis process.

There are two key reasons for maintaining pressure/temperature on the shell side of the boiling water reactor during standby mode:
If the methanol catalyst is exposed to low temperatures, there will be risk of wax condensing inside the catalyst causing catalyst deactivation.
If the reactor cools down and is heated up again during restart and this procedure is carried out often there will be risk of fatigue stress. By maintaining the boiling water pressure constant and thereby keeping the temperature variation for the reactor to a minimum during standby mode fatigue stress need not to be considered for the mechanical design.

Therefore, there is a need for a reliable and effective auxiliary heating that can be used in the event of disruptions in the methanol synthesis process, to ensure the efficient and continuous operation of the process. The present invention aims to provide such a system, as will be described in detail below.

The invention provides a process for the preparation of methanol comprising the steps of
(a) preparing a hydrogen feedstock by electrolysis
(b) providing a carbon oxide feedstock in periods of operating the electrolysis in step (a)
(c) mixing at least part of the hydrogen feed and carbon oxide source consisting of carbon monoxide and/or carbon dioxide feed to obtain a methanol synthesis gas;
(d) adjusting the molar content of hydrogen, carbon monoxide and/or carbon dioxide from step (c) to a module M of (H2−CO2)/(CO2+CO) to between 1.9 and 2.2
(e) converting the methanol synthesis gas in one or more boiling water reactors to methanol;
in periods without operating the electrolysis in step (a)
(f) interrupting the converting of the methanol synthesis gas in the one or more boiling water reactors by heat exchange with boiling water, wherein in step (f) the one or more boiling water reactors are heated by one or more auxiliary heaters to maintain boiling of the water in the one or more boiling water reactors.

In an embodiment of the invention, the one or more boiling water reactors are heated in step (f) by a common auxiliary heater.

In an embodiment, the one or more auxiliary heaters are electrical heaters. The electrical heaters are preferably arranged as part of the riser/downcomers system to the one or more boiling water reactor.

The riser/downcomers are pipes or channels that run alongside the reactor core. The primary function of the riser/downcomers is to supply coolant water from the upper part of the reactor vessel to the bottom, by natural circulation where it enters the core.

In an embodiment, a common electrical heater is arranged as part of steam drum of the methanol reactors and heat circulated through the boiling water reactors by circulation pump.

In an embodiment, heat input of the electrical heaters is controlled by adjusting the power input to maintain the pressure in the water side of the boiling water reactor.

In an embodiment, the electrical heaters are used for heating up the boiling water reactor during start-up.

An advantage of this embodiment is that an ejector typically used in conventional boiling water reactors during start-up of the reactor is superfluous.

In an embodiment, the electrolysis is operated with renewable electricity.

In an embodiment, the electrolysis is performed in a solid oxide electrolysis unit.

In an embodiment, the carbon oxide source is pure CO2 with a concentration of >95%, more preferable >99%.

In an embodiment, the carbon dioxide is biogenic and/or anthropogenic carbon dioxide. Biogenic carbon dioxide refers to carbon dioxide (CO2) that is released into the atmosphere through natural biological processes and the burning of biomass (such as wood or agricultural residues).

Anthropogenic carbon dioxide emissions refer to the release of carbon dioxide (CO2) into the atmosphere as a result of human activities. These emissions primarily stem from the burning of fossil fuels (such as coal, oil, and natural gas) for energy production, transportation, and industrial processes.

In an embodiment, CO2 is partially converted to CO by the electrolysis or in a separate electrolysis.

In an embodiment, at least part of the methanol is further converted to gasoline or jet fuel

1. Process for the preparation of methanol, comprising the steps of:
(a) preparing a hydrogen feedstock by electrolysis;
(b) providing a carbon oxide feedstock in periods of operating the electrolysis in step (a);
(c) mixing at least part of the hydrogen feed and carbon oxide source consisting of carbon monoxide and/or carbon dioxide feed to obtain a methanol synthesis gas;
(d) adjusting the molar content of hydrogen, carbon monoxide and/or carbon dioxide from step (c) to a module M of (H2−CO2)/(CO2+CO) to between 1.9 and 2.2;
(e) converting the methanol synthesis gas in one or more boiling water reactors to methanol;
in periods without operating the electrolysis in step (a):
(f) interrupting the carbon oxide feed in step (b) resulting in stop of the methanol formation in one or more methanol reactors,
wherein in step (f) the one or more boiling water reactors are heated by one or more auxiliary heaters to maintain boiling of the water in the one or more boiling water reactors.

2. The process of claim 1, wherein the one or more boiling water reactors are heated in step (f) by a common auxiliary heater.

3. The process of claim wherein the one or more auxiliary heaters are electrical heaters.

4. The process of claim 3, wherein the one or more electrical heaters are arranged in riser/downcomers system of the one or more boiling water reactor.

5. The process of claim 3, wherein a common electrical heater is arranged as part of steam drum of the methanol reactors and heat circulated through the boiling water reactors by a circulation pump.

6. The process of 3, wherein heat input of the electrical heaters are controlled by adjusting the power input to maintain the pressure in the water side of the boiling water reactor.

7. The process of claim 1, wherein the electrical heaters are used for heating up the boiling water reactor during start-up.

8. The process of claim 1, wherein the electrolysis is operated with renewable electricity.

9. The process according to claim 1, wherein the electrolysis is performed in a solid oxide electrolysis unit.

10. The process according to claim 1, wherein the carbon oxide source is pure CO2 with a concentration of >95%.

11. The process according to claim 1, wherein the carbon dioxide is biogenic and/or anthropogenic.

12. The process according to claim 1, wherein CO2 is partially converted to CO by the electrolysis or in a separate electrolysis.

13. The process according to claim 1, wherein at least part of the methanol is further converted to gasoline or jet fuel.
